(19) United States
(12) Patent Application Publication
(10) Pub. No.: US 2022/0032288 A1
(43) Pub. Date: Feb. 3, 2022

(54) VIRAL AND BIOCHEMICAL EARLY DETECTION TEST KITS

(71) Applicant: Smart Ink Technology Corp., Jenkintown, PA (US)

(72) Inventors: Auguste Jacques, Philadelphia, PA (US); Veonous Martin Jacques, Philadelphia, PA (US); Diane Renee Rhynes, Hockessin, DE (US)

(21) Appl. No.: 17/388,695

(22) Filed: Jul. 29, 2021

Related U.S. Application Data
(60) Provisional application No. 63/058,274, filed on Jul. 29, 2020.

(51) Int. Cl. B01L 3/00; G01N 33/569

(52) U.S. Cl. CPC B01L 3/5029; B01L 2300/0829; G01N 33/56983

(57) ABSTRACT

A test kit for detecting viral or biochemical infections is disclosed comprising a housing, at least one test well in the housing comprising a test material for detecting and indicating the presence of a virus, biochemical agent or antibodies in a test sample, and a control material adjacent the housing comprising a damage indicating material, a taggant material or a track and trace element. A method of detecting viral or biochemical infections using the test kit is also disclosed. The method includes contacting a test material with a test sample, and observing any change in appearance of the test material to determine the presence or absence of the virus, biochemical agent or antibodies.

### CROSS-REFERENCE TO RELATED APPLICATION

This application claims the benefit of U.S. Provisional Patent Application No. 63/058,274 filed Jul. 29, 2020, which is incorporated herein by reference.

### FIELD OF THE INVENTION

The present invention relates to viral and biochemical early detection test kits.

### BACKGROUND INFORMATION

Detection of viral and biochemical infections is well known. However, a need exists for simple and early detection.

### SUMMARY OF THE INVENTION

An aspect of the present invention is to provide a test kit for detecting viral or biochemical infections comprising a housing, a first test well in the housing comprising a first test material for detecting and indicating the presence of a virus, biochemical agent or antibodies in a first test sample, and a control material adjacent the housing comprising a damage indicating material, a taggant material or a track and trace element.

Another aspect of the present invention is to provide a method of detecting viral or biochemical infections using the test kit. The method comprises contacting the first test material with the first test sample, and observing any change in appearance of the first test material to determine the presence or absence of the virus, biochemical agent or antibodies.

These and other aspects of the present invention will be more apparent from the following description.

### BRIEF DESCRIPTION OF THE DRAWINGS

FIG. 1 is a perspective view and

FIG. 2 is a top view of a test kit applicator.

FIG. 3 is a top view of the test kit applicator vacuum sealed/airtight showing applicator has not been opened.

FIG. 4 is a top view of the test kit applicator after opening exposing control well to oxygen.

FIG. 5 is a top view of the test kit applicator after control well is exposed to oxygen activating the tamper indicating ink creating a color change to the control pad.

FIGS. 6 and 7 are top views of the test kit applicator when mucosa, blood, or saliva is applied to the reagent/antigen on the test pad, where the sample indicates a positive presence of the antibody causing the dye to change from colorless to Blue or any designated color.

FIGS. 8-10 are top views of the test kit applicator when mucosa, blood, or saliva is applied to the reagent/antigen on the test pad, where the sample indicates a positive presence of a viral infection causing the dye to change from colorless to Red or any designated color.

FIG. 11 is an exploded side view of a test kit applicator in accordance with an embodiment of the invention.

FIG. 12 is a top perspective view of the upper portion of a test kit applicator in accordance with an embodiment of the invention.

FIG. 13 is a top perspective view of the lower portion of a test kit applicator in accordance with an embodiment of the invention.

FIG. 14 is a partially schematic side sectional view showing a portion of a layered packaging material in accordance with an embodiment of the present invention.

FIG. 15 is a partially schematic side sectional view showing a portion of a layered packaging material in accordance with another embodiment of the present invention.

FIG. 16 is a partially schematic side sectional view showing a portion of a layered packaging material with track and trace elements and damage indicating materials in accordance with an embodiment of the present invention.

FIG. 17 is is a partially schematic side sectional view showing a portion of a layered packaging material with track and trace elements and damage indicating materials in accordance with another embodiment of the present invention.

### DETAILED DESCRIPTION

The present invention relates to early detection viral/biochemical test kits. More specifically, the present invention pertains to a portable viral/biochemical test kit that is suitable for home use. The test kit may be adapted to perform an immunoassay to measure the presence of antibodies/cytokines/biomarkers in various types of samples including mucosa, blood, saliva, tears, pus, fecal matter, urine, vaginal secretion, semen, and other human and animal secretions, thereby detecting presence of the antibody and/or the presence of the virus. The test kit may be used to test for Coronaviruses, HIV, HPV, Hep C, Ebola, meningitis, H1N1 and other viruses, bacteria, fungus, immune system disorders, and the like. If a sufficient level of antibodies/cytokines/biomarkers are detected in the mucosa, blood, or saliva sample, the test kit can visually indicate the test results. The test kits may include immunoassays that measure the presence of antibodies/cytokines/biomarkers, reagents and antigens. The test kits may include tamper-evident, taggant, or other control functions to ensure that the test kits have not been damaged or tampered with prior to use and/or to provide track-and-trace capabilities.

In accordance with embodiments of this invention, there is provided an early detection on-site test kit characterized in that materials contained therein allow a visual color change detection system to determine tampering, as well as, measure the presence of antibodies/cytokines/biomarkers in the test samples. Further features of the invention provide a tampering detection system that gives visual indication via color change, exposure to air to indicate viability of test kit.

The invention still further provides a method of tamper detection where an applicator is enclosed in an airtight/vacuum sealed packaging system. Once the package seal is opened, punctured, breached, or any way exposed to oxygen, a control well may change from no color to a designated color, which indicates the test kit has not been opened before intended use.

Still further features of the invention may provide reagents including antigens on a test pad that measure presence of cytokines/biomarkers when mucosa, blood, or saliva is applied directly onto the pad. This test slot may be indicative of the presence of the antibody, rather than being positive for a virus or biochemical.

The invention may also provide reagents/antigens on a test pad that measures the presence of antibodies when mucosa, blood, or saliva is applied directly onto the pad, to determine the positive presence of a viral infection. A viral infection includes, but is not limited to, influenza, coronavirus, Covid-19, acute bronchitis, the common cold, rhinovirus or other upper respiratory infections.

Further features of the invention provide the dye to be activated when the antibody binds with the antigens specific for viral infection including but not limited to CD2, CD3, CD14 and CD 40. When the concentration of specific viral antibodies in the mucosa, blood, or saliva exceeds a predetermined threshold, the test pad may display a color change. Conversely, the test window may be adapted to show a color change if the concentration of the antibodies falls below the predetermined threshold. Additionally, the control pad may be adapted to display a visual color change to indicate that the test was completed correctly, regardless of whether antibodies are or are not present in the mucosa, blood, saliva sample.

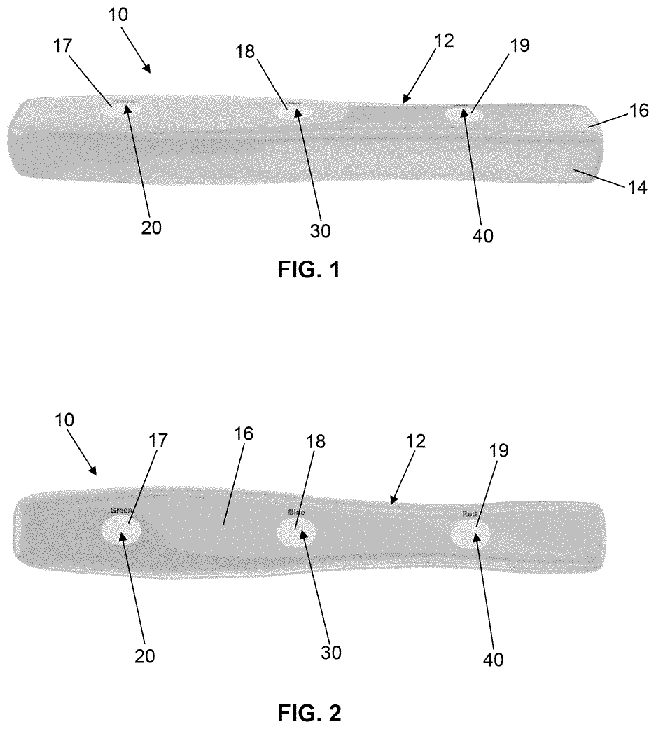

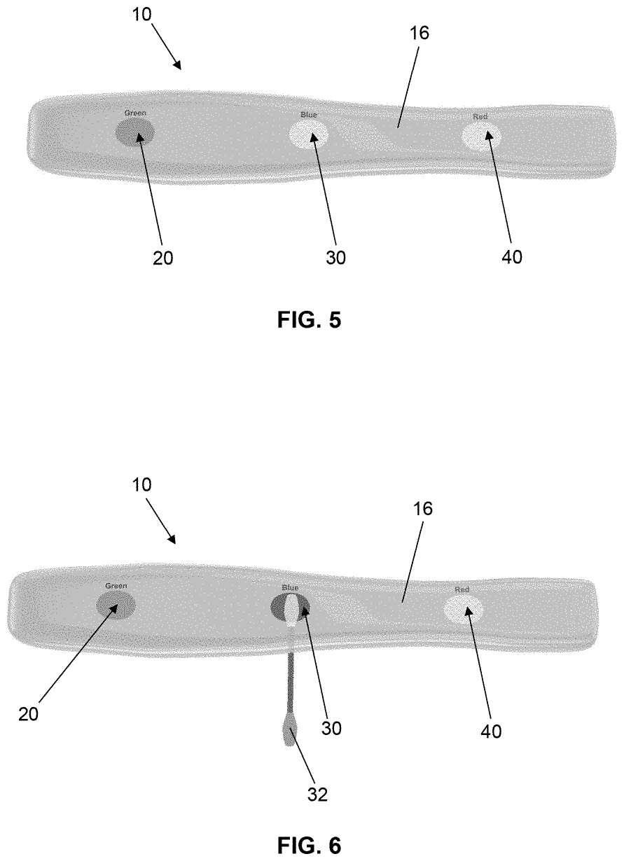

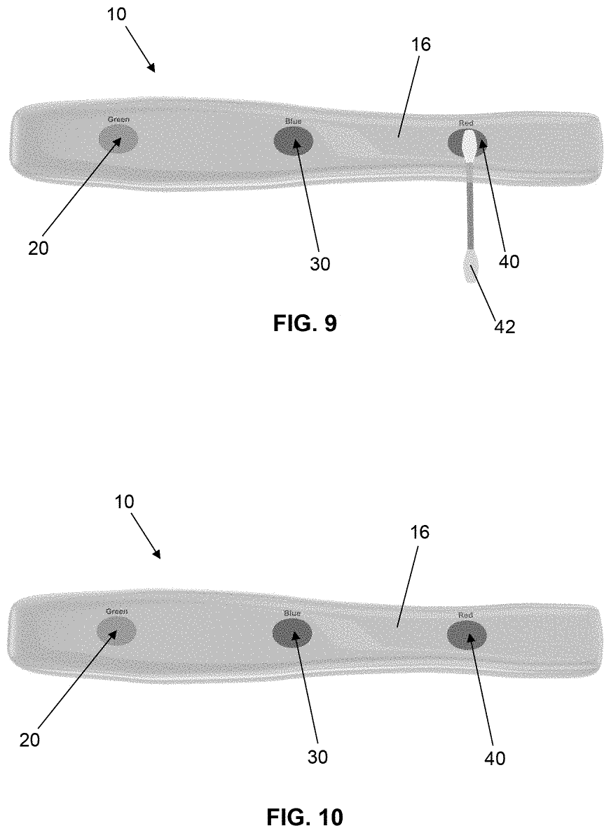

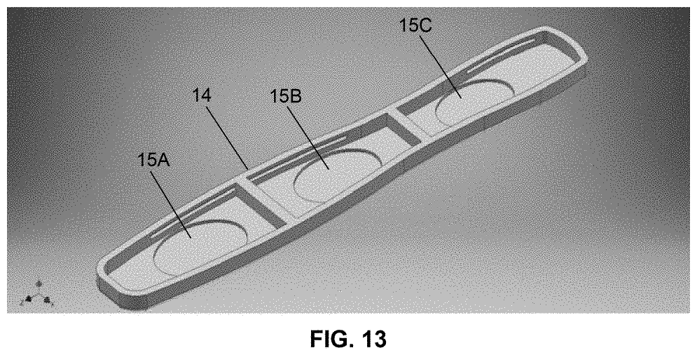

FIGS. 1-13 illustrate a test kit applicator 10 in accordance with an embodiment of the present invention. The test kit applicator 10 includes a housing 12 having a base 14 and a top 16. As shown most clearly in FIG. 13, the base 14 may include separate compartments 15A, 15B and 15C. As shown in FIGS. 1, 2 and 12, the top 16 includes three test openings 17, 18 and 19.

A control well 20 is provided in the test opening 17 of the top 16 above the control well compartment 15A. A first test well 30 is provided in the test opening 18 of the top 16 above the first test compartment 15B. A second test well 40 is provided in the test opening 19 of the top 16 above the second test well compartment 15C. Although the wells 20, 30 and 40 are circular in the figures, any other shape such as oval, square, rectangular, or the like.

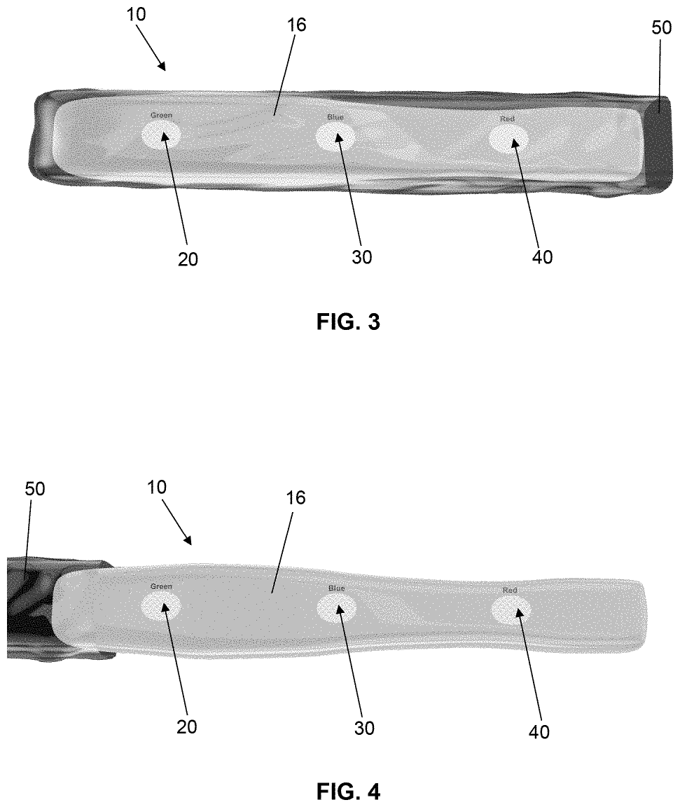

As shown in FIGS. 3 and 4, prior to use of the test kit applicator 10, a sealed wrapper 50 may surround the test kit applicator 10, and can be removed to expose the control well 20, first test well 30, and second test well 40. The sealed wrapper 50 may be evacuated and/or at least partially filled with an inert gas prior to opening of the wrapper.

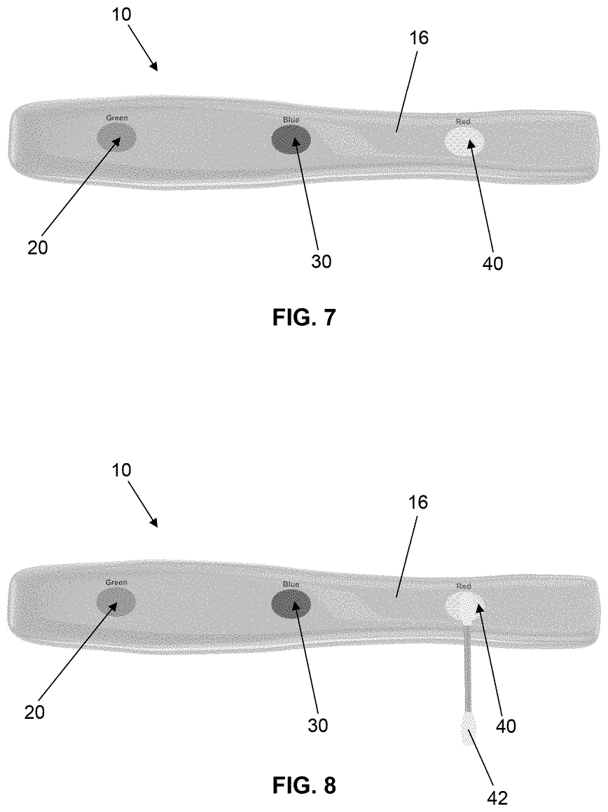

As shown in FIGS. 6 and 7, a first swab 32 may be placed against the first test well 30 in order to test for viral, biochemical or other agents.

As shown in FIGS. 8-10, a second swab 42 may be placed against the second test well 40 to detect the presence of viral, biochemical or other agents.

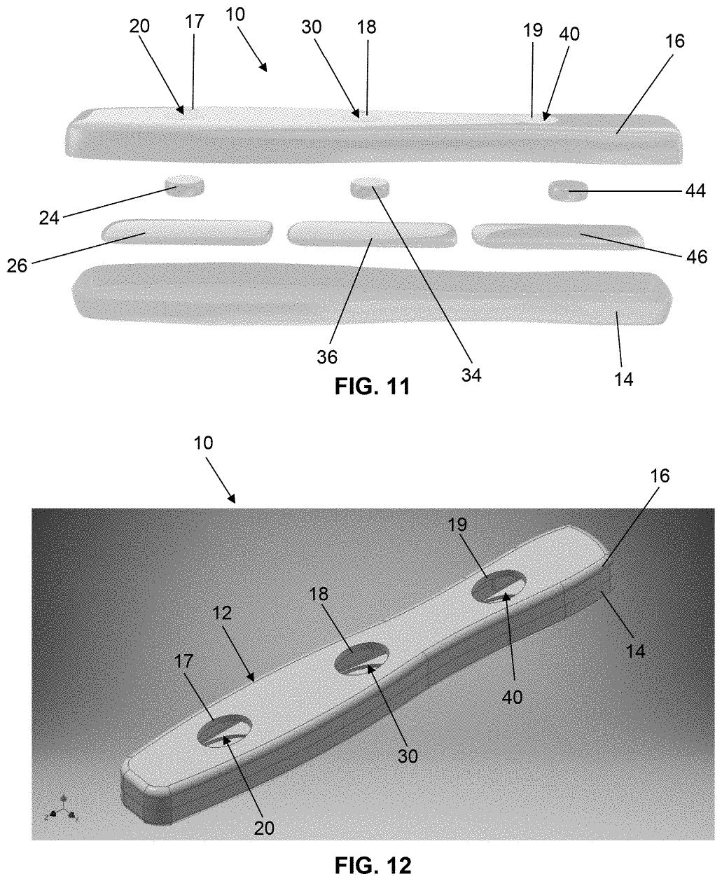

As shown in FIG. 11, the control well 20 may include a control pad 24 and an underlying control material 26. Although the control pad 24 and control material 26 are shown as separate components in the embodiment of FIG. 11, it is to understood that they may be combined into a single component, for example, a single control pad containing an oxygen-indicating material such as methylene blue.

As shown in FIG. 11, the first test well 30 includes a first test pad 34 and an underlying first test material 36, and the second test well 40 includes a second test pad 44 and underlying second test material 46. However, a single pad may be used in each of the first and second test wells 30 and 40.

ELISA-enzyme-linked immunosorbent assay may be used in the first test well 30 and/or in the second test well 40. The ELISA test involves an enzyme (a protein that catalyzes a biochemical reaction) and an antibody-Ab or antigen-Ag (an immunologic molecule) to form an Ab or Ag reaction to provide a positive result. If no reaction then there is a negative result. When enzymes bound to another substance as an indicator a color change can be caused.

Test components may include a microtiter plate that has a solid phase substrate (target protein, antigen) and a known concentration fixed to the plate that when exposed to an Ab that has an indicator attached (dye for color change or enzyme-labeled Ab) that can produce a color change.

The control well 20 may include an oxygen sensitive material, or other damage indicating materials, such as color-changing materials disclosed in U.S. Pat. No. 10,029,841, which is incorporated by reference herein. In addition, a taggant material and/or track and trace elements may be used in or near the control well, such as taggants disclosed in U.S. Pat. No. 10,029,841, and taggants and track and trace elements disclosed in U.S. application Ser. No. 16/042,416, which is incorporated by reference herein.

The damage indicating material contained in the control well 20 may comprise an absorption-based species that produces a visible color change caused by chromogenic chemistry that involves oxidation by molecular oxygen. Examples include methylene blue, resorufin, resazurin, thiazine, thionines, oxazine, azine, triphenylmethane, indophenol, indigo, thioindigo, pyridinium viologen, and quinone-based species. The damage indicating material may also comprise luminescence-based species such as polycyclic aromatic hydrocarbons, polypyridyl complexes, metalloporphyrins, including platinum and palladium complexes, cyclometallated complexes, and other luminescent metal complexes such as lead, aluminum, copper, gold, europium, terbium, molybdenum, and the like. Other damage indicating material species include fifflerenes, fluorescent polymers, and modified polymeric materials containing absorption-based or luminescence-based species described above. Additional responsive materials may be non-toxic phosphorous pigmentation, or any alternative oxygen sensitive reactive dyes or any dyes that change color when exposed to oxygen, extreme heat, or excessive pressure, compression or tension.

The reactive/responsive species of the damage indicating material can be incorporated into a variety of formulations including inks, gels, plastics, composites and the like. Ink formulations include resin/binder variations such as gelatin, cellulosics such as hydroxyethylcellulose (HEC), ethyl cellulose, cellulose acetate, polyvinylalcohol (PVA), polyvinylpyrolidone (PVP), polyamides, polyurethanes, polyethylene oxide (PEO), poly acrylates such as polymethyl methacrylate (PMMA), polymethacrylate, and polystyrene or modified polystyrenes. The ink formulations may include inks typically used on plastic films and vacuum packaging films, or for pad printing, flexo printing, gravure printing, dot matrix style printing, steganography printing, and the like. Semiconductor variations include oxides of titanium, tin, tungsten, zinc and/or mixtures thereof. Semiconductor size variations include submicron diameter, sub 50 nm diameter, and sub 10 nm diameter. Sacrificial electron donor variations may be a mild reducing agent, amine such as sodium salt of ethylenediaminetetraacetic acid (NaEDTA) or triethanolamine (TEOA), saccharide such as glucose or fructose, antioxidant such as ascorbic acid or citric acid, or other easily oxidizable species such as glycerin or oxidizable polymer such as polyvinylalcohol (PVA). Additive variations include solubility modifiers such as surfactants, permeability modifiers such as silicones, and modifiers for oxygen transmissibility rate (OTR) or water vapor transmissibility rate (WVTR). Examples of additives include moisture absorbers, oxygen scavengers, microwave susceptors and antimicrobials.

In certain embodiments, anti-counterfeiting taggant materials may be added to the control material contained in the control well 20 in order to authenticate the product in order to protect against counterfeiting. Known types of taggants may be added to the damage indicating material. For example, taggant particles or compositions may be added to coatings, inks and adhesives, or embedded in packaging layers and/or labels. Types of anti-counterfeiting taggant materials include infrared (IR) responsive particles and inks, ultraviolet (UV) responsive particles and inks, secure pigments, metachromic materials, color-shift materials, thermochromic materials, and the like. A non-limiting example of commercially available taggants that may be added to, or used in association with, the damage indicating materials of the present invention is a particulate taggant material sold under the designation Microtaggant by Microtrace, LLC. Other types of commercially available taggant materials include SunGuard inks sold by Sun Chemical Corporation. When taggant particles or compositions are added to the present damage indicating coatings, they may typically be present in amounts ranging from a minimal detectable trace amount up to 5 or 10 weight percent of the coating, or more. For example, taggants may comprise from 0.001 to 5 weight percent, or from 0.01 to 2 weight percent, or from 0.1 to 1 weight percent of the material.

In certain embodiments, track and trace elements may be added to the control material contained in the control well 20, added to the packaging outer layer, added to packaging inner layer, added to packaging design (pocket), added in proximity of the damage indicating material, added in proximity of the packaging layers or pocket, created by formation of unique patterns or layers or generated via methods such as blockchain that can capture, link and secure all information about the product. The track and trace elements may be used to identify, authenticate, retain information/data, record location, report location or provide live location tracking over a range distances (short range contact to long range global positioning). Types of track and trace elements include taggant materials as described above, and may also include machine readable codes such as Quick Response (QR) codes, bar codes, active radio frequency identification (RFID), passive RFID, Bluetooth low energy (BLE), WiFi, GPS and the like. Types of track and trace elements also include real-time live tracking systems (RTLS). RTLS can be based on a wide variety of systems including active RFID, passive RFID, infrared (IR), optical, ultrasound, WiFi, Bluetooth and the like and combinations thereof Non-limiting examples of commercially available track and trace elements are embeddable RFID transponders from HID in low (LF), high (HF) and ultrahigh (UHF) frequencies and various form factors and sizes. Other types of commercially available track and trace elements include RFID inlay designs commercially available from Avery Dennison for operation in the UHF, HF bands and near field communication (NFC). For GPS track and trace elements, a commercially available GPS transceiver with a micro-battery or other suitable power source may be used. Different types of track and trace elements can be used in various combinations to meet the requirements for products. Blockchain technology can also generate track and trace capability for a product and/or substantially enhance capture, security, transparency and visibility of data from other track and trace technologies.

In addition to, or in place of, the damage indicating, taggant and/or track and trace materials being provided in the control well 20, the outer packaging may include damage indicating material, taggants and/or track and trace elements, as more fully described below.

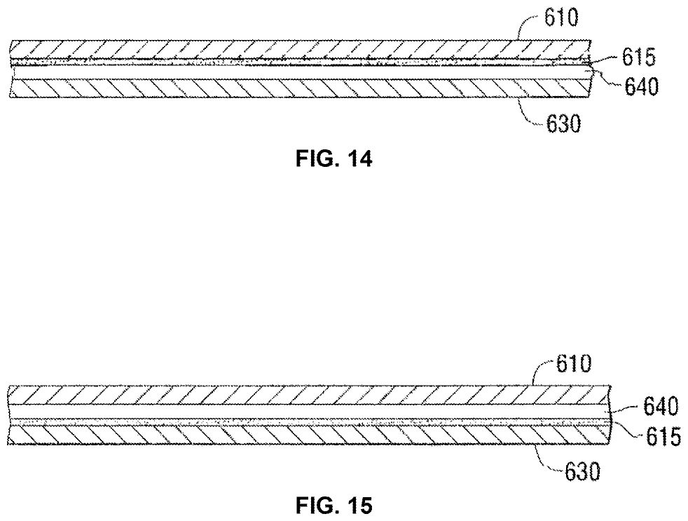

FIGS. 14 and 15 are partially schematic side sectional views illustrating various damage indicating packaging layers that may be incorporated in the packaging wrapper 50 in accordance with embodiments of the present invention. In FIG. 14, an outer layer 610 is provided with a layer of damage indicating material 615 applied thereto. An inner wrapper layer 630 is located adjacent to the outer layer 610 and damage indicating material 615. In certain embodiments, the inner wrapper layer 630 may not be adhered to the layer of damage indicating material 615, as shown by the gap 640 in FIG. 14. Although the gap 640 is shown as a physical spacing between the layers 630 and 615 in FIG. 14, it should be recognized that the gap may be closed such that the layers 630 and 615 contact each other. For example, when the space between the outer layer 610 and inner wrapper layer 630 is evacuated, the layer of damage indicating material 615 would typically contact the underlying inner wrapper layer 630. Alternatively, when the space between the outer layer 610 and inner wrapper layer 630 is filled with an inert or non-reactive gas, the pressure of the gas may result in the formation of a physical gap 640, as shown in FIG. 14.

The embodiment shown in FIG. 15 is similar to the embodiment of FIG. 14, except the layer of damage indicating material 615 is applied to the outer surface of the inner wrapper layer 630 rather than the inner surface of the outer layer 610.

In accordance with embodiments of the present invention, the various inner wrapper layers and outer layers may be made of any suitable materials such as polymeric films, foils, paper and the like. Some examples of polymeric layers include cellulosic materials, vinyl polymers such as polyvinyl alcohol and polyacrylates, polyolefins such as polyethylene, polyethylene terephthalate (PET), ethylene vinyl acetate copolymers, polyethylene, nylon (polyamide) and the like. The inner wrapper layers and outer layers may be made of the same or different materials. In certain embodiments, the inner wrapper layers may comprise foil coated with any of the aforementioned polymers, or such polymers alone. In certain embodiments, the outer layers may comprise polyethylene or the like, which may optionally be coextruded with nylon or the like.

In an embodiment of the invention, the test kit 10 is disposed in an inner wrapper, an outer wrapper surrounds the inner wrapper, and a layer of damage indicating material is applied to the inner surface of the outer wrapper. Alternatively, as described above, the outer surface of the inner wrapper may have the damage indicating material applied thereto. In both of these embodiments, the space between the inner and outer wrappers may be evacuated by any suitable type of vacuum source in order to remove gasses including oxygen from the space between the wrappers. In this embodiment, when the outer wrapper is punctured, torn or otherwise breached, air will fill the previously evacuated space between the inner and outer wrappers, thereby coming into contact with the relatively large surface areas of the wrappers, i.e., the outer surface of the inner wrapper will be exposed to air and the inner surface of the outer wrapper will be exposed to air. The presence of the damage indicating material on the inner surface of the outer wrapper and/or on the outer surface of the inner wrapper will thereby provide an indication that the outer wrapper has been punctured, torn or otherwise breached and that air has entered the space between the wrappers. As an alternative to evacuating the space between the inner and outer wrappers, the inner space may be at least partially filled with an inert or non-reactive gas such as nitrogen or the like that does not cause the damage indicating material to react and change colors.

In accordance with an embodiment of the present invention, dual-wrapper arrangements as described above may be made by providing the test kit 10 in the inner wrapper, followed by applying the outer wrapper around the inner wrapper. For example, the outer wrapper may be provided as a pre-formed pocket in which the inner wrapper containing the test kit 10 is inserted, followed by sealing of the open end of the outer wrapper. As discussed above, before, during or after the sealing operation, the space between the inner and outer wrappers may be evacuated and/or filled with a non-reactive gas. As another example, separate sheets of outer wrapper material may be placed on opposite sides of the inner wrapper containing the test kit 10, following by sealing of the peripheral edges of the outer wrapper layers together to thereby seal the inner wrapper and test kit 10 within the outer wrapper. Again, the space between the inner and outer wrappers may be evacuated and/or filled with a non-reactive gas during the sealing operation. Such operations, in which the test kit 10 is first sealed in the inner wrapper followed by sealing an outer wrapper around the inner wrapper, may be conducted contemporaneously with each other, e.g., the inner and outer wrappers may be applied in the same manufacturing operation. Alternatively, pre-packaged test kits may be modified by applying the outer wrapper at a different time or location, e.g., at a different facility from the original product manufacturing location.

In accordance with another embodiment of the present invention, a single test kit wrapper is provided with multiple laminated layers in which at least one of the layers contains the damage indicating material. For example, a layer of damage indicating material may be sandwiched between inner and outer polymeric layers to provide a composite wrapper structure with damage indicating capabilities. As another example, a layer of damage indicating material may be applied on the inner surface of the single test kit wrapper. In this embodiment, the damage indicating material layer may be exposed to the test kit, and the damage indicating material must be non-reactive with the test materials of the test kit 10 or any other liquids or gasses contained within the wrapper, and the damage indicating layer must not damage the test kit components or vice versa.

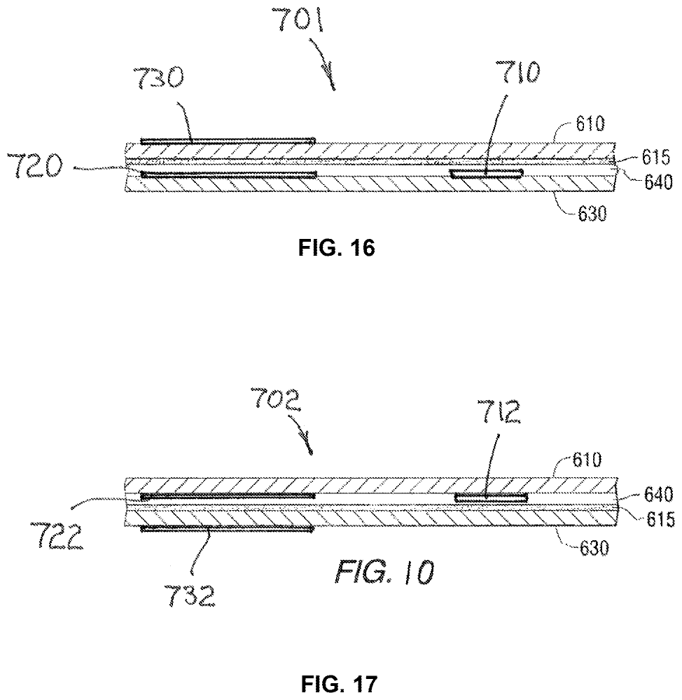

FIGS. 16 and 17 are partially schematic side sectional views of layered packaging materials 701 and 702 in accordance with embodiments of the present invention. In the embodiment shown in FIG. 16, the layered packaging material 701 includes damage indicating elements 610, 615, 630 and 640 similar to the embodiment shown in FIG. 14. In addition, the packaging 701 of FIG. 16 includes track and trace elements 710, 720 and 730 located on or in the inner wrapper layer 630 or the outer layer 610. Although multiple track and trace elements 710, 720 and 730 are shown at different locations with respect to the inner wrapper layer 630 and outer layer 610 in FIG. 16, it is to be understood that only one such element may be used, or two such elements may be used.

In the embodiment shown in FIG. 17, the layered packaging material 702 includes damage indicating elements, 610, 615, 630 and 640 similar to the embodiment shown in FIG. 15. In addition, the packaging 702 of FIG. 17 includes track and trace elements 712, 722 and 732 located on or in the inner wrapper layer 630 or the outer wrapper layer 610. Although multiple track and trace elements 712, 722 and 732 are shown at different locations with respect to the inner wrapper layer 630 and outer layer 610 in FIG. 17, it is to be understood that only one such element may be used, or two such elements may be used.

Examples of specific first test materials 36 and/or second test materials 46 that may be used in the first and second test wells 30 and 40 are described below.

Saliva samples are suitable for detection of some enzymes, antimicrobial agents, and other secreted analytes. ELISA kits are commercially available and use pre-coated polystyrene plates, detection antibodies and usually all the chemicals needed to perform an

ELISA test. ELISA tests are generally good and accurate test. They are considered highly sensitive and specific and favored over other methods used for detection of substances in the body. The test is straight forward and easier to perform which makes it ideal for in home testing use.

Types of ELISA tests include: direct ELISA—attachment of an antigen to the polystyrene plate followed by enzyme-labeled Ab that react with antigen and substrate that can be measured; indirect ELISA—attachment of an Ag to a polystyrene plate followed by an unlabeled or primary Ab followed by an enzyme-labeled Ab that can react w/both Ab and substrate; sandwich ELISA—measures reactions in the substrate; and competitive ELISA—results in a decrease in the substrate signal generated but gives a good highly specific result.

Any functional assay using serum can in theory be performed with mucosal secretions, depending of course on the concentration of the active factor. Below are some examples of tests that can be performed with mucosal antibodies. Apart from antibodies, any soluble factor can be measured in a secretion, as long as a sensitive enough assay exists. For instance, cytokines have been successfully detected in saliva or cervical washes. However, one has to be aware of potential drawbacks when measuring cytokines, due to their high sensitivity to proteolysis or denaturation, in particular in secretions.

A number of samples may be used to diagnose viruses, including whole saliva, gingival crevicular fluid, oral swabs of mucosal tissue, dental plaque, oral biopsy specimens and volatiles in breath. Studies reported in the literature typically involved the use of whole saliva or another oral fluid obtained by means of an adsorbent collector. Many of the tests on the market involve the use of oral mucosal transudate (OMT) obtained by swabbing the buccal mucosa and tongue. This sample is rich in antibodies and contains surface pathogens. Saliva, as collected from the salivary glands, consists primarily of secretory IgA (sIgA), whereas OMT contains a mixture of sIgA, IgG and IgM. Thus, OMT provides a richer source of antibodies, including those directed against bacterial and viral pathogens, and it is relatively more concentrated in oral surface pathogens and antigens derived from those pathogens. We should note that investigators in many research studies performed analyses on whole saliva clarified by centrifugation, and these results may differ from those for fluid collected with an oral swab. A large number of viruses can be detected in oral samples by using an antigen, an antibody or nucleic acid targets. For diagnosis of severe and high-mortality infections such as Ebola or rabies, antibody assays may be less useful because the infected patient may not survive long enough to go through seroconversion.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

As used herein, “including,” “containing” and like terms are understood in the context of this application to be synonymous with “comprising” and are therefore open-ended and do not exclude the presence of additional undescribed or unrecited elements, materials, phases or method steps. As used herein, “consisting of” is understood in the context of this application to exclude the presence of any unspecified element, material, phase or method step. As used herein, “consisting essentially of” is understood in the context of this application to include the specified elements, materials, phases, or method steps, where applicable, and to also include any unspecified elements, materials, phases, or method steps that do not materially affect the basic or novel characteristics of the invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. In this application and the appended claims, the articles “a,” “an,” and “the” include plural referents unless expressly and unequivocally limited to one referent.

Whereas particular embodiments of this invention have been described above for purposes of illustration, it will be evident to those skilled in the art that numerous variations of the details of the present invention may be made without departing from the invention as defined in the appended claims.

What is claimed is:

1. A test kit for detecting viral or biochemical infections comprising:
a housing;
a first test well in the housing comprising a first test material for detecting and indicating the presence of a virus, biochemical agent or antibodies in a first test sample; and
a control material adjacent the housing comprising a damage indicating material, a taggant material or a track and trace element.

2. The test kit of claim 1 further comprising a test well in the housing containing the control material.

3. The test kit of claim 2, wherein the test material comprises a damage indicating material that changes appearance upon exposure to oxygen.

4. The test kit of claim 2, wherein the control material comprises a taggant material comprising taggant particles.

5. The test kit of claim 2, wherein the control material comprises a track and trace element comprising a machine readable code or radio frequency identification element.

6. The test kit of claim 1, further comprising packaging surrounding the housing containing the control material.

7. The test kit of claim 6, wherein the test material comprises a damage indicating material that changes appearance when the packaging is opened.

8. The test kit of claim 1, wherein the first test material detects and indicates the presence of a virus in the test sample.

9. The test kit of claim 8, wherein the virus comprises a coronavirus, HIV, HPV, Hep C, Ebola, meningitis or H1N1.

10. The test kit of claim 8, wherein the virus comprises a coronavirus.

11. The test kit of claim 1, wherein the first test material detects and indicates the presence of a biochemical aunt in the test sample.

12. The test kit of claim 1, wherein the first test material detects and indicates the presence of antibodies in the test sample.

13. The test kit of claim 1, further comprising a second test well in the housing comprising a second test material for detecting and indicating the presence of a virus, biochemical agent or antibodies in a second test sample.

14. The test kit of claim 13, wherein the first and second test materials are different.

15. The test kit of claim 14, wherein the first test material detects and indicates the presence of a virus in the first test sample, and the second test material detects and indicates the presence of an antibody associated with the virus in the second test sample.

16. The test kit of claim 13, further comprising a test well in the housing containing the control material.

17. The test kit of claim 1, further comprising a first test swab structured and arranged to carry the first test sample and to contact the first test well.

18. The test kit of claim 13, further comprising a second test swab structured and arranged to carry the second test sample and to contact the second test well.

19. The test kit of claim 1, wherein the first test material is contained in or under a first test pad in the first test well.

20. The test kit of claim 13, wherein the second test material is contained in or under a second test pad in the second test well.

21. A method of detecting viral or biochemical infections using the test kit of claim 1, the method comprising:
contacting the first test material with the first test sample; and
observing any change in appearance of the first test material to determine the presence or absence of the virus, biochemical agent or antibodies.
